SEQUENCE LISTING (2 sequences)

SEQ ID NO: 1 - DNA, 51 bp, Artificial Sequence
  allosteric agonist of the receptor CXCR4
gcgtcgttgtggttgtcttacagatgtccatgtagattcttcgaatctcac

SEQ ID NO: 2 - DNA, 51 bp, Artificial Sequence
  allosteric agonist of the receptor CXCR4
cggtcggtgatgttgtcttacagatgtccatgtagattcttcgaatctcac

(19) United States
(12) Patent Application Publication
(10) Pub. No.: US 2005/0065064 A1
(43) Pub. Date: Mar. 24, 2005

(54) Identification of allosteric peptide agonists of CXCR4

(76) Inventors: Elias Lolis, Fairfield, CT (US); Aristidis Sachpatzidis, New Haven, CT (US); Henrik G. Dohlman, Chapel Hill, NC (US); John Manfredi, Salt Lake City, UT (US)

Correspondence Address: Chris Kolefas, 1017 Edgewood Lane, Fort Lee, NJ, 07024, US

(21) Appl. No.: 10/637,911

(22) Filed: Aug. 8, 2003

Related U.S. Application Data
(60) Provisional application No. 60/402,474, filed on Aug. 9, 2002.

(51) Int. Cl. A61K 38/16

(52) U.S. Cl. 514/2

(57) ABSTRACT

The chemokine receptor CXCR4 is a co-receptor for T-tropic strains of HIV-1. A number of small molecule antagonists of CXCR4 are in development, but all are likely to lead to adverse effects due to the physiological function of CXCR4. To prevent these complications, allosteric agonists may be therapeutically useful as adjuvant therapy in combination with small molecule antagonists. A synthetic cDNA library coding for 160,000 different SDF-based peptides was screened for CXCR4 agonist activity in a yeast strain expressing functional receptor. Peptides that activated CXCR4 in an autocrine manner induced colony formation. Two peptides, designated RSVM and ASLW, were identified as novel agonists that are insensitive to the CXCR4 antagonist AMD3100. In chemotaxis assays using the acute lymphoblastic leukemia cell line CCRF-CEM, RSVM behaves as a partial agonist and ASLW as a superagonist. The superagonist activity of ASLW may be related to its inability to induce receptor internalization. In CCRF-CEM cells, the two peptides are also not inhibited by another CXCR4 antagonist, T140, or the neutralizing monoclonal antibodies 12G5 and 44717.111. These results suggest that alternative agonist binding sites are present on CXCR4 that could be screened to develop molecules for therapeutic use.

### FIELD OF THE INVENTION

The present invention relates generally to allosteric agonists and more specifically to allosteric agonists which provide beneficial biological activity with a reduction in the harmful side-effects often associated with primary site agonists.

### BACKGROUND INFORMATION

CXCR4 is a member of the chemokine receptor family of G protein-coupled receptors. A number of mechanisms for receptor activation of G protein-coupled receptors have been proposed (1,2). For example, small molecule agonists bind within the transmembrane helices and cause activation. Larger molecules bind to specific sites on the extracellular surface of the receptor leading to a conformational change that is transmitted to an intracellular Gapy complex. This results in an exchange of GTP for GDP in the Gα protein, dissociation of Gα from the Gβγ complex, and activation of downstream signal transduction pathways. Competitive receptor antagonists bind to the same or overlapping agonist site, and prevent the physiological agonist from activating the receptor. In principle, allosteric modulators that bind to different sites on G protein-coupled receptors should have no effect on activity except in the presence of receptor agonists or antagonists (3). Allosteric agonists or antagonists could also bind at different sites and induce biological activity.

SDF-1α is the sole physiological agonist for the receptor CXCR4. Deletion of either of the SDF or CXCR4 genes in mice is lethal, with developmental defects in the cerebellum, the heart, the gastrointestinal tract, and hematopoiesis (4-6). In adults, SDF-1α is constitutively secreted from virtually all cell types and is involved in the migration and development of hematopoietic cells. CXCR4 is an important target for HIV-1 drug discovery due to its additional role as an HIV-1 co-receptor (7). However, inhibition of the HIV-1 binding site, which corresponds to the agonist binding site, is likely to lead to severe adverse effects. Allosteric agonists or modulators of CXCR4 activity would be therapeutically useful, but have not yet been identified. Such agents could preserve the function of CXCR4 and minimize adverse effects expected from the use of competitive receptor antagonists in HIV-1 therapy.

The first 7 or 8 N-terminal residues of SDF-1α that precede the first of four cysteines, which define the chemokine superfamily, are not evident in the three-dimensional structure (8,9). In virtually all chemokine structures, the N-terminal sequence preceding the first cysteine is not observable in either NMR or X-ray structures and is presumed to be flexible. The flexibility of the N-terminal sequence in over a dozen known chemokine structures suggests that this mobility is important for the biological function of these proteins (10). Mutation or deletion of the residues at the N-terminal sequence of chemokines usually leads to a functional change from receptor agonism to antagonism (11,12). Despite a large number of studies on chemokine structure and function, how these proteins interact with their G protein-coupled receptors remains to be elucidated. A two-step process has been proposed involving the binding of a chemokine to its receptor followed by placement of the N-terminal flexible loop of the chemokine on another site of the receptor, leading to its activation. In this model, the N-loop, consisting of the first 20-30 residues of chemokines, provides most of the specificity for the receptor. For SDF-1α, N-terminal peptides of 9-17 amino acids are weak agonists of CXCR4 (13).

A number of mammalian receptors have been successfully expressed in Saccharomyces cerevisiae and shown to couple to the native or modified versions of G proteins leading to activation of the pheromone response pathway (14-22). To identify allosteric agonists or modulators of CXCR4 activity, we adapted a yeast expression system that produces functional CXCR4. S. cerevisiae is known to possess one of two G protein-coupled receptors, Ste2p and Ste3p, for each haploid cell type. In yeast Gβγ activates a MAP kinase pathway resulting in transcription of genes containing a pheromone responsive element (PRE) in their promoter, growth arrest, and mating of the two different haploid yeast strains. We describe a genetically modified yeast strain that allowed us to identify both normal and allosteric agonists. We focus on the characterization of two peptide agonists, designated RSVM and ASLW, that are resistant to the small molecule CXCR4 antagonists AMD3100 and T140 and the neutralizing antibodies 12G5 and 44717.111. These peptides may serve as lead compounds for development of drugs that could be used in conjunction with antagonists for anti-HIV therapy. The data also illustrate the presence of alternative agonist binding sites for screening non-peptide allosteric agonists.

### BRIEF DESCRIPTION OF THE DRAWINGS

FIG. 1. (A) The normal pheromone pathway in Sacchramocyces cerevisiae. (B) Genetic modifications created to identify new agonists. Genes that are inactivated are indicated by an X. Expression of His3 and lacZ is under the control of the Fus1 promoter, which contains a pheromone response element (PRE).

FIG. 2. Functional expression of CXCR4 in yeast. (A) Comparison of colony induction in histidine-deficient plates as a function of 3-amino-1,2,4-triazole (AT) concentration by HIS3⁻/CXCR4⁺/SDF⁺ yeast strains (-▪-) versus parental HIS3⁻ strains (-●-). Growth is due to stimulation of the pheromone pathway through activation of CXCR4 by SDF-1α. (B) Induction of β-galactosidase activity of HIS3⁻/CXCR4⁺ yeast cells by SDF-1α (100 nM), the enantiomeric D-SDF-1α as a negative control (100 nM), and the 14-mer N-terminal peptide (1 μM). (C) The β-galactosidase activity induced by 100 nM SDF-1α is inhibited by AMD3100 (1 μM), a known CXCR4 antagonist. AMD3100 does not effect the basal level of the pheromone-responsive Fus1 promoter.

FIG. 3. CXCR4 dependence of each peptide activity established in yeast. The background strain (first bar) expresses only the receptor and reporter plasmids. Its β-galactosidase activity is significantly increased when it is transformed with the (A) RSVM- or (B) ASLW-expressing plasmid (second bar). The presence of the reporter and peptide-expressing plasmids does not induce this effect in the absence of CXCR4 (third bar).

FIG. 4. Chemotactic properties of agonists on CCRF-CEM acute lymphoblastic leukemia cells. Concentration-response chemotactic activity of (A) RSVM and (B) ASLW. A plateau in chemotactic index of either peptide could not be observed due to insolubility of both peptides above the indicated concentrations. (C) Chemotactic activity of RSVM in the presence of SDF-Fc indicates that RSVM has properties of a partial agonist. Mixtures of agonists (D) ASLW and SDF-Fc or (E) ASLW and RSVM have additive chemotactic effects. (F) Chemotactic activities of the two mutant peptides, RSVM (200 μM), and ASLW (100 μM) compared to the activities of the wild-type agonists SDF-Fc (100 nM), SDF-1α (5 nM) and N-terminal 17-mer peptide (20 μM). All ligands were used at their optimal concentration. B=Background chemotaxis.

FIG. 5. Effect of various antagonists on the chemotactic activity of CXCR4 agonists on CCRF-CEM cells. (A) Sensitivity of SDF-Fc and the wild-type 17-mer peptide to AMD3100. Resistance of (B) RSVM and (C) ASLW agonist activity to inhibition by AMD3100. Resistance of (D) RSVM and (E) ASLW to inhibition by the CXCR4 antagonist T140.

FIG. 6. Effect of CXCR4-specific monoclonal antibodies on the chemotactic activity of the peptides. Lack of neutralization of (A) RSVM and (B) ASLW activity by 12G5. 44717.111 shows the same lack of effect on the (C)RSVM and (D) ASLW peptide activities on CCRF-CEM cells.

FIG. 7. (A) Chemotactic activity of full-length, mutant [RSVM]SDF-Fc protein on CCRF-CEM cells. Sensitivity of chemotactic activity of [RSVM]SDF-Fc to the CXCR4 antagonists (B) AMD3100, (C) T140, and the CXCR4 neutralizing monoclonal antibodies (D) 12G5 and (E) 44717.111.

FIG. 8. Effect of SDF-Fc and the RSVM and ASLW peptides on CXCR4 surface levels detected by flow cytometry. FL1-H on the x-axis represents fluorescence intensity. In (A), (B), and (C) the basal levels are indicated by bold lines, the agent-treated cells by dashed lines, the isotype and secondary antibody controls by dotted and straight lines respectively. (A) SDF-Fc (1 μM) causes a clear decrease of the basal CXCR4 surface levels. (B) RSVM (200 μM) causes a similar effect. (C) The effect is not shared by ASLW (100 μM). (D) RSVM has a concentration-dependent response. (E) The lack of a significant response for ASLW is evident at different concentrations.

### DETAILED DESCRIPTION

Materials and Methods

Vector Construction and Strains.

The initial S. cerevisiae strain was CY12946 (MATα FUS1p-HIS3 GPA1Gαi₂₍₅₎ canI farIΔI442 his3 leu2 sst2Δ2 ste14::trp1::LYS2 ste3Δ1156 tbt1-1 trp1 ura3) (23). Except for the specific chimeric Ga subunit expressed, CY12946 is genetically similar to CY1141, reported in Klein et al. (18), with two differences. First, the two strains express different Gα subunits. Specifically, CY12946 expresses a chimeric Gα subunit (GPA1Gαi₂₍₅₎) in which the C-terminal 5 amino acids of the yeast Ga subunit, GPA1, are replaced by the C-terminal 5 residues of Gαi₂. In contrast, as described by Klein et al. (18), CY1141 expresses a chimeric Ga subunit (GPA1₍₄₁₎Gαi₂) in which the N-terminal 33 residues of Gαi2 are replaced by the 41 N-terminal residues of GPA1. We have used CY12946 in the current report because GPA1Gαi₂₍₅₎ is more efficient than GPA1₍₄₁₎Gαi₂ in coupling CXCR4 to the pheromone response pathway. The use of GPA1Gαi₂₍₅₎ necessitated deletion of SST2, which down-regulates the pheromone response pathway by accelerating the GTPase activity of GPA1. Thus, the second difference between CY12946 and CY1141 is the presence of an sst2 deletion allele in CY12946. All plasmids were 2μ-derived and contained a REP3 element for autonomous replication in S. cerevisiae and AmPR for selection in Escherichia coli. The plasmid Cp4181 contains the CXCR4 gene under the control of the constitutive phosphoglycerate kinase (PGK1) promoter and a LEU2 selectable marker. Plasmid Cp1584 possesses the FUS1-lacZ construct that allows production of β-galactosidase when the pheromone pathway is stimulated. It also contains the TRP1 selectable marker. Cp6160 expresses full length wild-type SDF-1α in yeast under the control of the constitutive alcohol dehydrogenase (ADH1) promoter. The α-factor signal sequence is cloned at the 5′-end of the SDF sequence to allow secretion of the mature polypeptide. The vector also has the URA3 selectable marker allowing transformed cells to grow in media deficient in uracil. For expression of the 17-mer peptide library, Cp6293 was used. This URA3+plasmid was derived from Cp6160 and contains a frameshift in the SDF-1α sequence so that no functional protein is expressed unless an insert is appropriately cloned. The cDNA coding for 17-mer peptides was cloned using the restriction sites HindIII and Acc65I, which also results in the secretion of the mature peptide. S. cerevisiae strains YAS1, YAS2, YAS3 were constructed by transforming CY12946 with Cp1584, Cp1584/Cp4181 and Cp1584/Cp4181/Cp6160, respectively.

Media.

All basic media were prepared essentially as described (24). In cases where plasmid selection was necessary, media that lacked uracil, leucine, or tryptophan or an appropriate combination of these nutrients were employed. Library screening was performed in plates that lacked histidine and contained 3-amino-1,2,4-triazole (Sigma), which suppresses the basal level of histidine biosynthesis due to the activity of the FUS1 promoter.

Library Construction and Tranformation.

An oligonucleotide library coding for 17-mer peptides, in which the first four codons were randomized, was chemically synthesized by the Yale University Keck facility. The library was made with oligonucleotides that used the triplet NNK (N is any nucleotide and K is either G or T) to encode the first four amino acids (25). Codons 5 through 17 were from the wild type N-terminal sequence of SDF-1α. The modified genetic code used for randomization allows a more balanced representation of all amino acids while at the same time limiting the number of possible stop codons to one. The double-stranded oligonucleotides containing the random nucleotides were constructed so that the new sequence is in-frame with the prepro-α signal sequence after ligation. The sequences of the two primers were: 5′-GCCGTCAGTAAAGCTTGCTTAAGCGTNNKNNKNKNNKTTGTCTTACAGATGTCCA TGTAGATTCTTCGAATCTCACTGAGGTACCAGTCTGTGACGC-3′ and 5′-GCGTCACAGACTGGTACCT-3′, where N is an equimolar mixture of A, G, C and T, K represents an equimolar mixture of G and T, and sequences in bold are either HindIII or Acc65I sites. Formation of the semi-randomized gene was performed as described in (25). Briefly, one nmol each of the two single-stranded oligonucleotides were annealed by combining in 200 μl of 40 mM Tris-HCl (pH 7.5), 10 mM MgCl₂, 0.1 mg/ml of BSA, and 0.5 mM dithiothreitol, heating the mixture to 65° C. for 20 min, and then slowly cooling to 37° C. The partially double-stranded primers were filled in by adding 5 μl of 10 mM deoxynucleoside triphosphates and 3 μl of Sequenase (13U/ml, United States Biochemical Corp.) followed by successive incubations at 37° C. for 30 min and 65° C. for 20 min. The DNA product was digested with HindIII and Acc65I, and gel-purified. The Cp6293 vector was similarly HindIII/Acc65I digested and gel-purified. Different insert/vector ratios were used in overnight ligations at 14° C. followed by transformation of E. coli XL-10 ultracompetent cells (Stratagene). Aliquots were removed and plated in LB-ampicillin plates to estimate library size; the remaining bacteria were added to 500 ml of LB-ampicillin for overnight growth at 37° C. for isolation of library DNA. Optimization of the insert:plasmid ratio resulted in 10⁸ ampicillin-resistant clones that should contain all of the possible nucleotide sequences. The quality of the library was evaluated by sequencing the peptide-encoding inserts in plasmids recovered randomly from bacterial clones. All inserts encoded heptadecapeptides, and the frequencies of amino acids at the randomized positions were not different from those predicted by chance alone. Yeast cells were transformed with the library DNA by using a modification of the lithium acetate method (26).

β-Galactosidase Assay.

The liquid beta-galactosidase assays were performed essentially as described in (24) or (27) with the following modification. After growth in the appropriate medium, 5 ml of cells displaying the agonist phenotype were centrifuged (2500 rpm for 5 min) and resuspended in 0.5 ml as described (28).

Peptide and Protein Production.

Peptides were synthesized and purified by the Keck Peptide Synthesis Facility at Yale. DMSO was used when necessary to facilitate dissolution of the lyophilized peptides. SDF-1α and its enantiomer were kind gifts of Gryphon Sciences (29). For expression of SDF-1α and its mutants, a mammalian vector pcDNA3.1 that coded for a fusion protein between SDF-1α and the Fc domain of antibodies were kindly provided by Drs. Qing Ma and Timothy Springer (Harvard University). HEK-293 cells were transformed by the calcium phosphate method, the media were collected, iodoacetamide was added to prevent aggregation from the Fc portion, and the protein was purified using Sepharose-Protein A affinity chromatography. For construction of mutants in the full-length protein, the Quikchange (Stratagene) site-directed mutagenesis kit was used and each mutation was confirmed by DNA sequencing.

Antibodies and CXCR4 Antagonists.

Neutralizing monoclonal antibodies 12G5 and 44717.111 were obtained from the NIH AIDS Research Program and R&D Systems, respectively. The FITC-linked rat anti-mouse IgG2a and the isotype-matched control antibodies were obtained by Pharmingen. T140 was a kind gift of Dr. Nobutaka Fujii of Kyoto University. A modification of the original method was used to synthesize AMD3100 (30). NMR and mass spectrometry were used to verify the synthesis.

Chemotaxis Assay.

The CCRF-CEM acute lymphoblastic leukemia cell line was previously shown to express CXCR4 and to migrate in response to SDF-1α (31). The procedure involved cells that were ˜70-80% confluent and were centrifuged at 1000 rpm for 5 min and resuspended at a concentration of ˜10⁷ cells/ml. The transwell system (Corning-Costar^{R}) was used for quantifying the chemotactic response. Six hundred μl of solution with different concentrations of a CXCR4 agonist were added to the bottom of the wells of 24-well culture plates. One hundred μl of the cell suspension was aliquoted in the transwell cups and inserted in the wells. The bottom of the cups is layered with a membrane that has a 5 μm pore size and allows cell movement towards the higher concentration of chemoattractant. The cells were incubated for 2h at 37° C., after which the migrated cells were collected and counted with an electronic particle counter (Coulter^{R}). In cases where the effect of neutralization by CXCR4 monoclonal antibodies or antagonists was examined, the inhibitor was added to the upper compartment along with the cells.

Flow Cytometry.

Flow cytometric analysis of cell subpopulations was performed using a FACS Vantage flow cytometer (Becton-Dickinson Immunocytometry Systems, San Jose, Calif.) for detection of CXCR4 surface expression with the 12G5 monoclonal antibody. An indirect staining protocol was used. Briefly, after a 90 min incubation with various agents, the cells were washed and resuspended in PBS. Nonspecific (Fc-mediated) binding was blocked using purified rat IgG. The primary antibody (12G5, a mouse IgG2a antibody) was added at 1 μg/ml for 45 min. After centrifugation the cells were resuspended in PBS and the secondary antibody (FITC-linked rat anti-mouse IgG2a) was added at 10 μg/ml for 45 min. The cells were fixed with 2% formaldehyde (in PBS) and used within 2 days for data acquisition. Mouse IgG2a was used as an isotype control. For the fluorescence measurements the cells were excited at 488 nm. The FITC fluorescence was collected through a 530/30 nm band pass filter. A minimum of 15,000 cells were examined for each sample. Analysis of data was performed using the CellQuest software (Becton-Dickinson).

Results

Functional Expression of Human CXCR4 in Yeast.

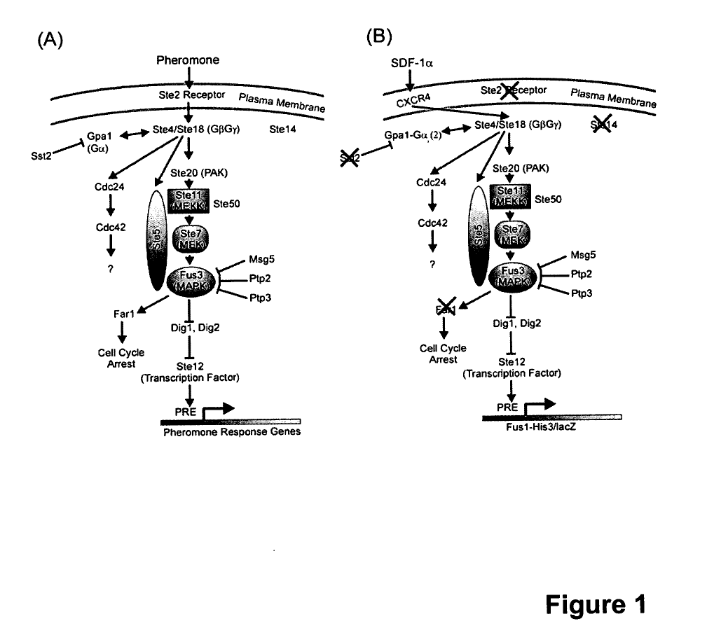

To identify allosteric peptide agonists or modulators of CXCR4, it was necessary to establish an assay that could isolate molecules with the desired properties from a large peptide library. The screen that was used consisted of two steps. The first step was to isolate peptide sequences from a synthetic cDNA library that would allow cells to grow only after activation of CXCR4. A second step was required that would segregate agonists that bind to the native active site from those that bind to allosteric sites. This step involved an agonist assay in the presence of a receptor antagonist. A number of genetic modifications made to S. cerevisiae allowed it to be used for monitoring the interactions of peptides or proteins with CXCR4 (FIG. 1). First, the endogenous α-factor G protein-coupled receptor was genetically disrupted. CXCR4 expression was achieved by its presence on an episomal plasmid. Second, the yeast Gα protein was modified such that its five C-terminal residues were replaced with those from human Gαi2, the Ga subunit known to mediate signaling by CXCR4. The resulting chimeric Ga subunit effectively couples CXCR4 to the yeast pheromone response pathway. Third, FAR1 was deleted, since its gene product mediates the growth arrest that normally results from activation of the pheromone response pathway (32). As a result, deletion of FAR1 dissociated pathway stimulation from growth arrest while still allowing CXCR4-dependent induction of pheromone responsive genes. Fourth, SST2, a negative regulator of GPA1 (the yeast Ga subunit), was deleted in order to enhance responsiveness to activated CXCR4. Finally, STE14 was deleted to dampen background activity of the pheromone response pathway.

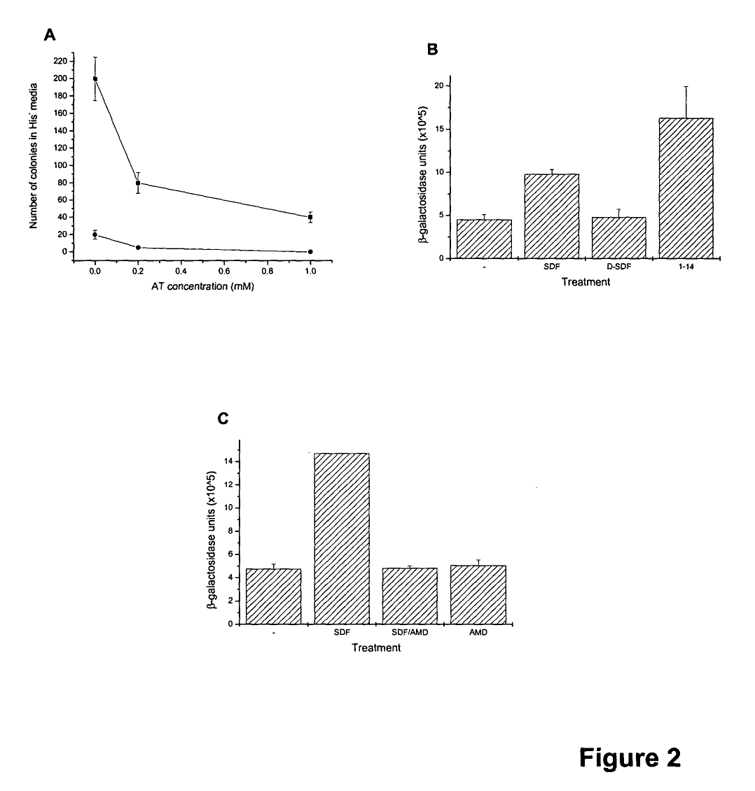

A number of genes for biosynthetic enzymes have been mutated to allow selection of the corresponding marker on a plasmid transformed into the yeast strain. The genomic HIS3 gene, coding for an enzyme essential for the biosynthesis of histidine, has been disrupted by an insertion. The wild-type HIS3 gene has been placed under the control of the pheromone-responsive FUS1 promoter; as a result activation of CXCR4 enables cells to grow in media lacking histidine. To reduce false positives due to any basal activity of FUS1-HIS3,3-amino-1,2,4-triazole, an inhibitor of the enzyme coded by HIS3, is present at low levels in the media during agonist selection. Endogenously expressed SDF-1α stimulates the production of histidine and allows the strain expressing CXCR4 to grow in the absence of this amino acid due to the FUS1-HIS3 gene (FIG. 2A). In a strain where FUS1-lacZ is also present, endogenous or exogenous SDF-1α induces an increase of β-galactosidase activity (FIG. 2B). Similar effects were observed in response to a peptide agonist comprised of the first fourteen amino acids (1-14) of the SDF-1α sequence that had been shown in mammalian cells to act as a weak partial agonist (33). To show specificity, we tested the activity of the enantiomer of the chemokine, D-SDF-1α, which showed no effect on the β-galactosidase assay (FIG. 2B). Furthermore, the CXCR4 antagonist AMD3100 (34) inhibited YAS2 colony formation (data not shown) and β-galactosidase activity in response to SDF-1α (FIG. 2C). The effects of SDF-1α are dependent on the expression of CXCR4, as a strain without the receptor (YAS 1) does not respond to SDF-1α, or the 14-mer peptide (data not shown).

Library Screening and Agonist Isolation.

The cDNA library encodes a 17-mer peptide with random amino acids at positions 1-4. At these positions the wild-type sequence is KPVS. The amino acid sequence for the remaining peptide (positions 5-17) retains the SDF-1α wild-type sequence. Transformation of strain YAS2 with the plasmid library yielded ˜10⁶ transformants. Each possible peptide is expected to be represented an average of approximately six times. The transformants were replica-plated enmasse to histidine-deficient medium containing 1 mM 3-amino-1,2,4-triazole. Originally ˜120 Ura⁺/His⁺ clones grew on these plates. The β-galactosidase activity of these colonies was measured and compared with strain YAS2, which expresses only CXCR4, or YAS3 that expresses SDF-1α with CXCR4. Forty one of these colonies displayed significant β-galactosidase activity. The active library plasmids were isolated, amplified in E. coli, and re-introduced into the YAS2 strain to confirm the CXCR4-mediated effects. From the 41 clones, 13 presented β-galactosidase activity that was plasmid-dependent. Two of these clones, predicted to express peptides with RSVM and ASLW sequences at their N-termini, showed resistance to AMD3100 in β-galactosidase assays and were selected for further characterization using chemically synthesized peptides.

CXCR4 Dependence of RSVM and ASLW Effect.

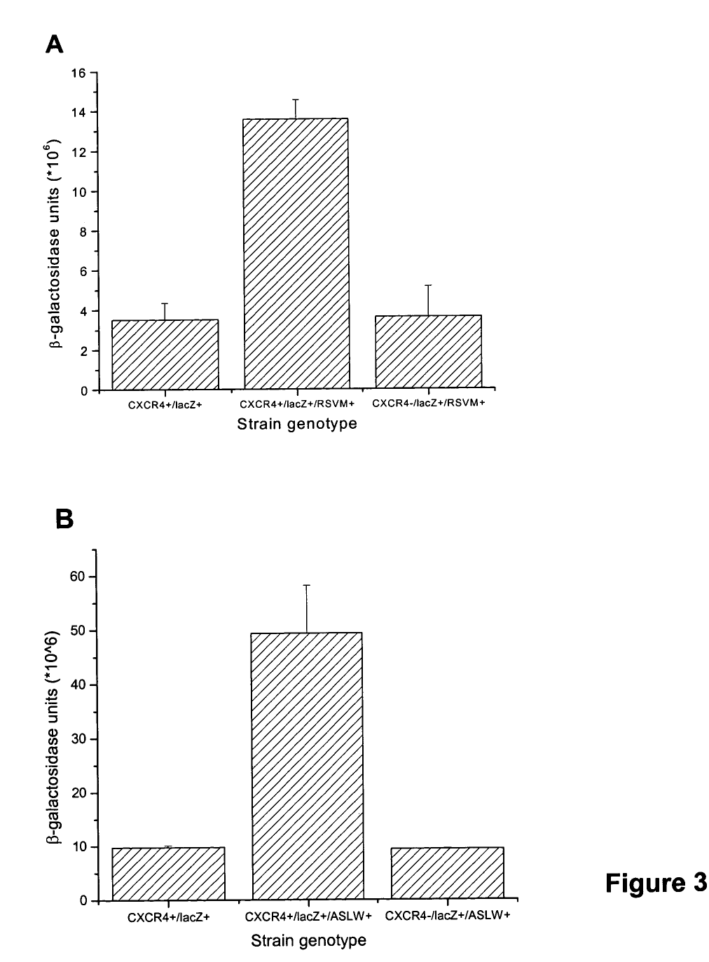

Initial experiments to test the effect of each exogenous peptide on the beta-galactosidase activity of CXCR4+yeast cells that contained the pheromone-responsive FUS1-lacZ construct were hampered by the toxicity of the peptides during the overnight exposure at the high concentrations (≧50M) required for testing. Therefore, we tested the CXCR4 dependence by the in vivo expression of the peptides in two different yeast strains, one that contained CXCR4 and another one that lacked this receptor. The results of these experiments are shown in FIG. 3. Endogenous expression of the peptides leads to a significant increase of β-galactosidase activity only in the strain that co-expresses CXCR4. Consequently, CXCR4 is required for activation of the pheromone pathway by the RSVM and ASLW peptides in yeast.

Peptide Assays.

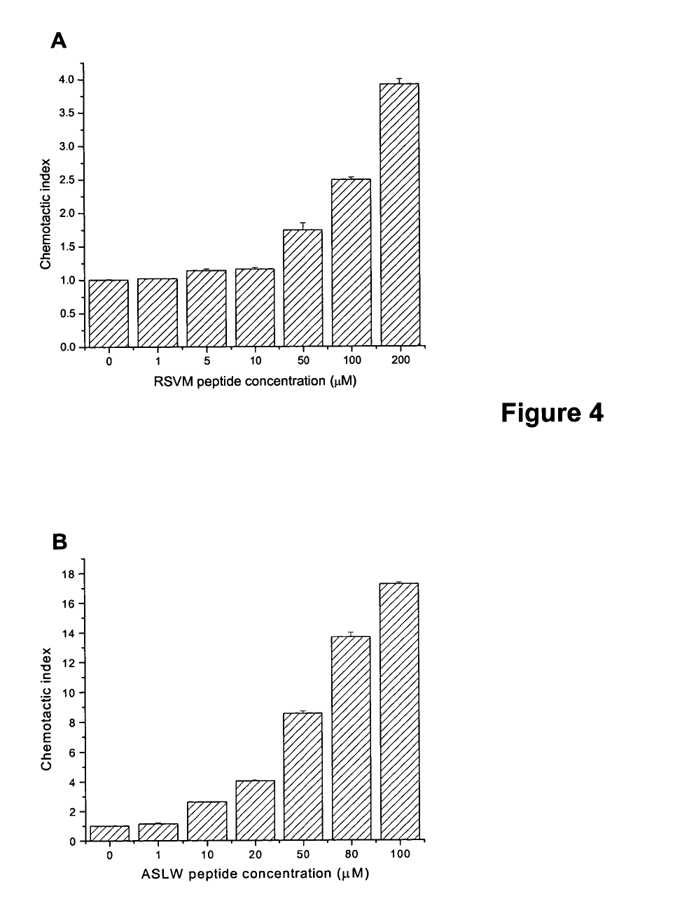

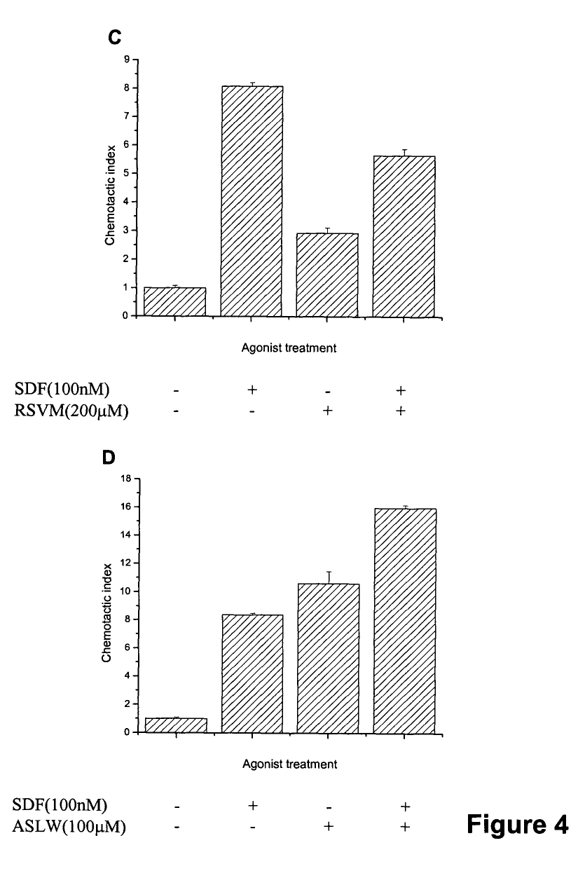

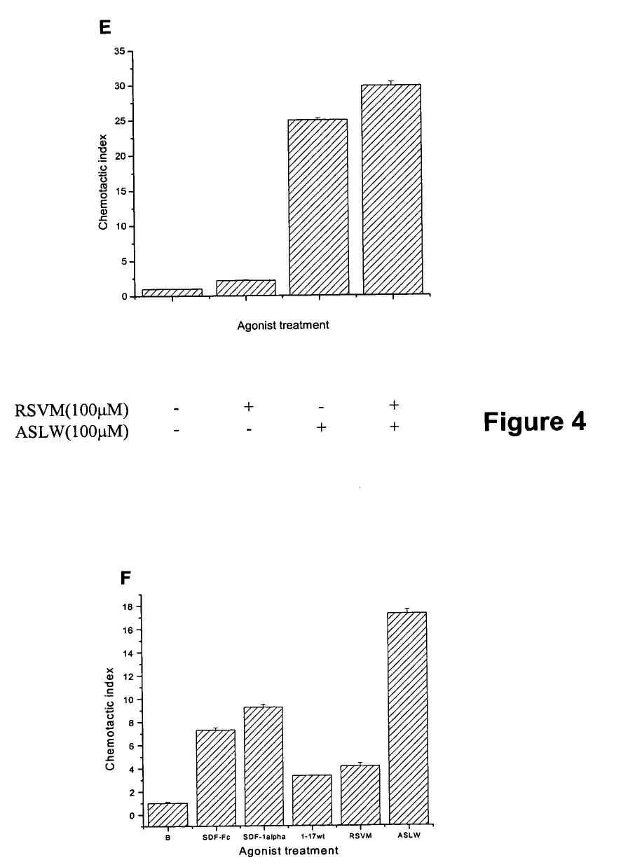

To verify that yeast screening provided peptides that possessed biological activity on mammalian cells, chemotaxis of the human leukemic T cell line CCRF-CEM was measured. FIGS. 4A and 4B display the concentration-response curves for the RSVM and ASLW peptides, respectively. The RSVM peptide has an apparent EC₅₀ of greater than 100 μM (FIG. 4A). Due to poor solubility of the peptides we have not been able to test concentrations higher than 200 μM in order to observe the typical bell-shaped concentration-response curve of most chemoattractants. The second peptide, ASLW, has an apparent EC₅₀ of greater than 60 μM (FIG. 4B) and displays superagonist activity, with a chemotactic index higher than the maximum observed with SDF-Fc. (SDF-Fc is a fusion protein between SDF-1α and the Fc region of an immunoglobulin that has similar agonist effects and is sometimes used instead of SDF-1α due to its stability.) Chemotactic effects induced by simultaneous stimulation of more than one agonist vary depending on which two agonists are used. The co-stimulation by SDF-Fc and RSVM leads to a chemotactic index that is about 30% lower than SDF-Fc alone (FIG. 4C). Thus, RSVM behaves as a weak partial agonist. However, the activities of SDF-Fc and ASLW are approximately additive (FIG. 4D). Chemotaxis in the presence of the two peptide agonists ASLW and RSVM is also additive (FIG. 4E). The relative chemotactic activities of all different CXCR4 agonists used in this study are shown in FIG. 4F at their optimal concentrations.

Effect of AMD3100, T140, 12G5, and 44717.111.

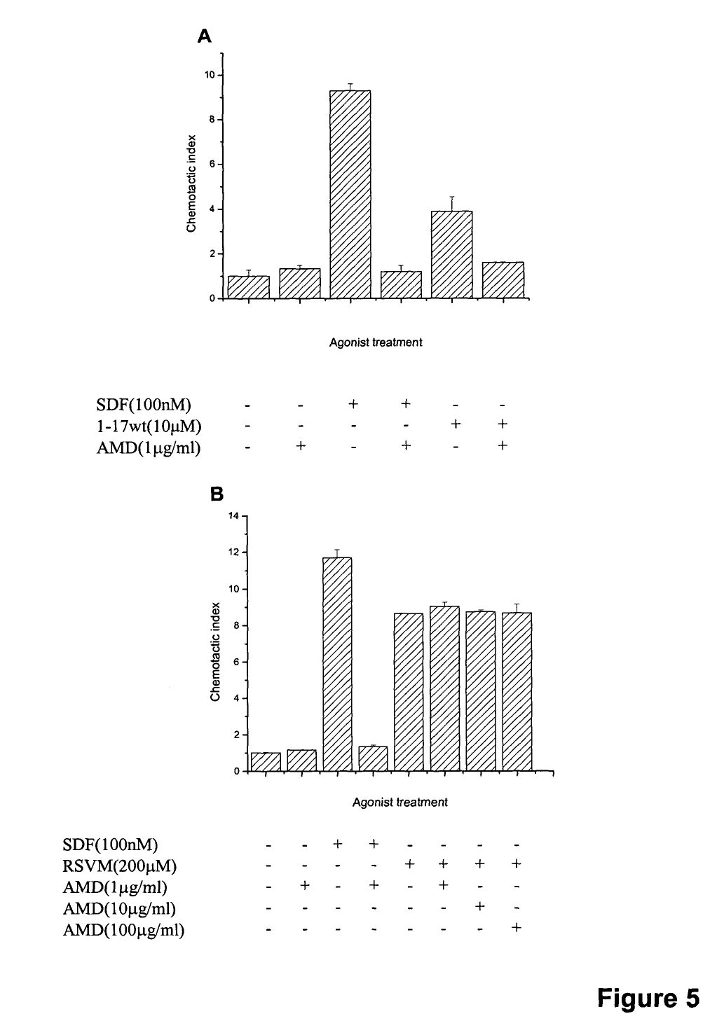

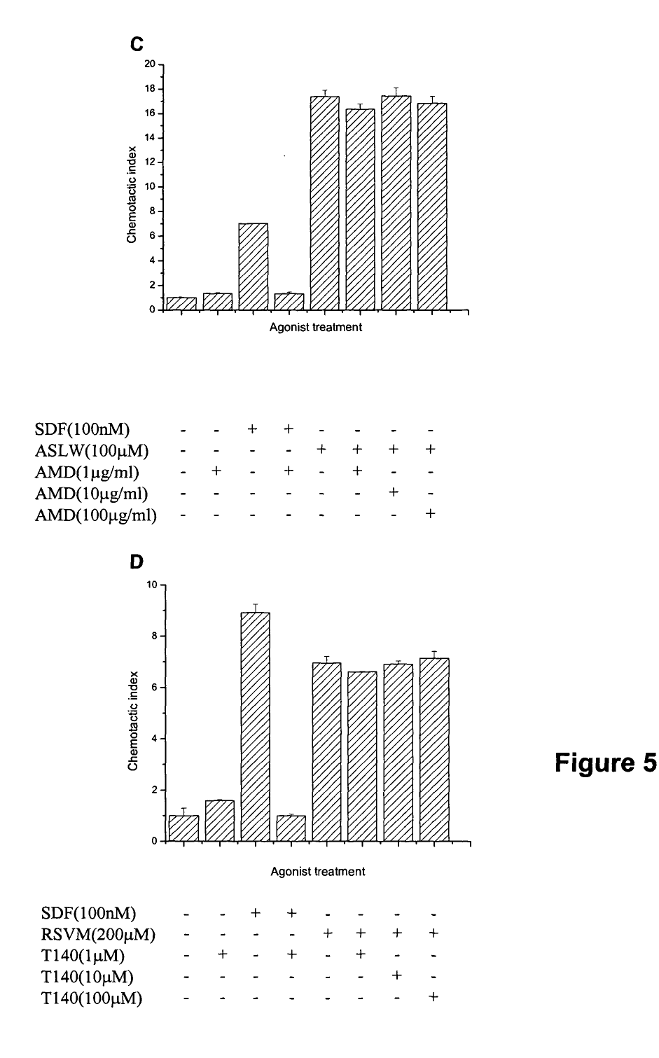

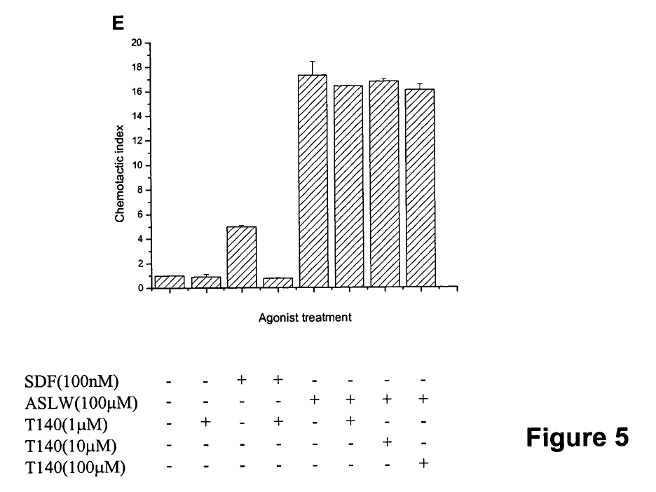

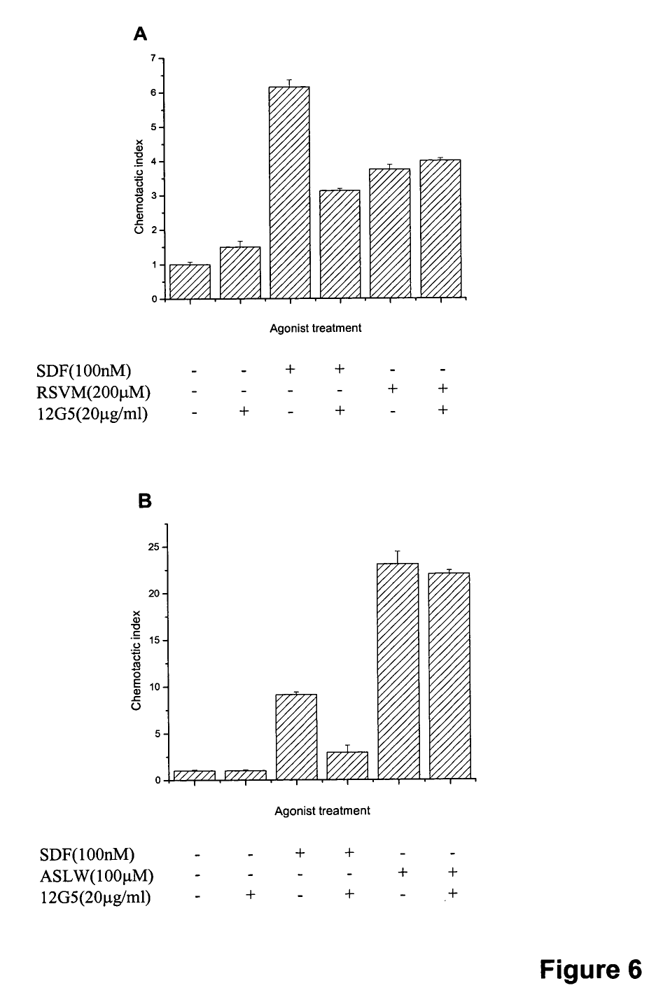

The chemotactic activity of SDF-Fc or the wild-type 17-mer is sensitive to AMD3100 (FIG. 5A). However, neither RSVM (FIG. 5B) nor ASLW (FIG. 5C) show any reduction in activity in the presence of AMD3100. The data suggest that AMD3100 and the peptides bind to different sites on the receptor. The unique agonist binding site, however, continues to activate CXCR4. Strong resistance to inhibition remains even at very high AMD3100 concentrations (100 μg/ml˜115 μM). This fact essentially excludes the possibility of a competitive interaction with the antagonist.

T140 is a 14-residue analogue of polyphemusin II from American horseshoe crabs that is also an antagonist of CXCR4 (35). The presence of this antagonist also does not inhibit CCRF-CEM chemotaxis in response to RSVM (FIG. 5D) or ASLW (FIG. 5E). Again, no significant reduction of the peptide activities is seen, even at T140 concentrations comparable to those of the agonists. Since AMD3100 and T134, a 14-residue analog of T140, do not compete for binding to CXCR4 (36), it is reasonable to expect that AMD3100 and T140 also do not compete with each other and therefore bind to different sites on CXCR4. These sites must sufficiently overlap with SDF-1α or HIV-1 gp120 in order to exert their antagonist effects. The experiments here indicate that RSVM and ASLW bind to sites that differ from and do not overlap with the AMD3100 and T140 sites.

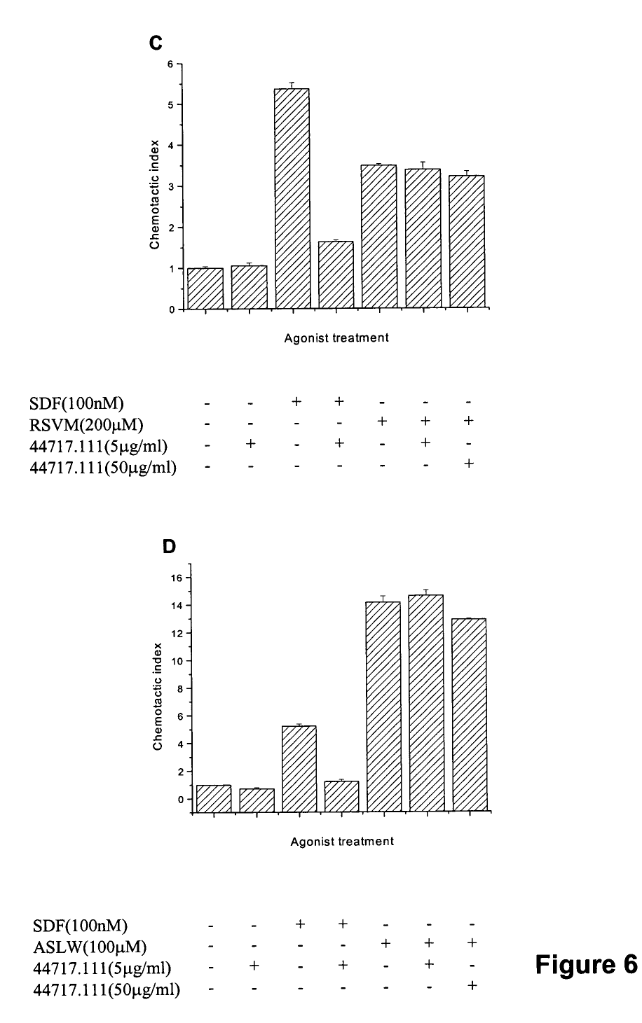

12G5 and 44717.111 are monoclonal antibodies to CXCR4 that prevent SDF-la mediated effects and HIV-1 infection of T-tropic cells (37,38). We examined the effect of co-incubation of each antibody with CCRF-CEM cells on the chemotactic activity of the peptides. FIG. 6A illustrates a substantial decrease in the chemotactic index for SDF-Fc in the presence of 12G5, but a very small effect of the antibody on RSVM. The same is observed for the ASLW peptide mutant (FIG. 6B). 12G5 binds an epitope on the first and second extracellular loop (39,40) that appears to overlap the AMD3100 binding site on the second extracellular loop and the adjacent transmembrane segment, TM4 (41). The epitope for 44717.111 has not yet been determined. However, as can be seen in FIGS. 6C and 6D, neither RSVM- nor ASLW-mediated chemotactic activity is significantly affected by the presence of 44717.111, even at a concentration (50 μg/ml) ten-fold higher than the one that almost completely abolishes the activity of SDF-Fc.

Full-Length Mutants.

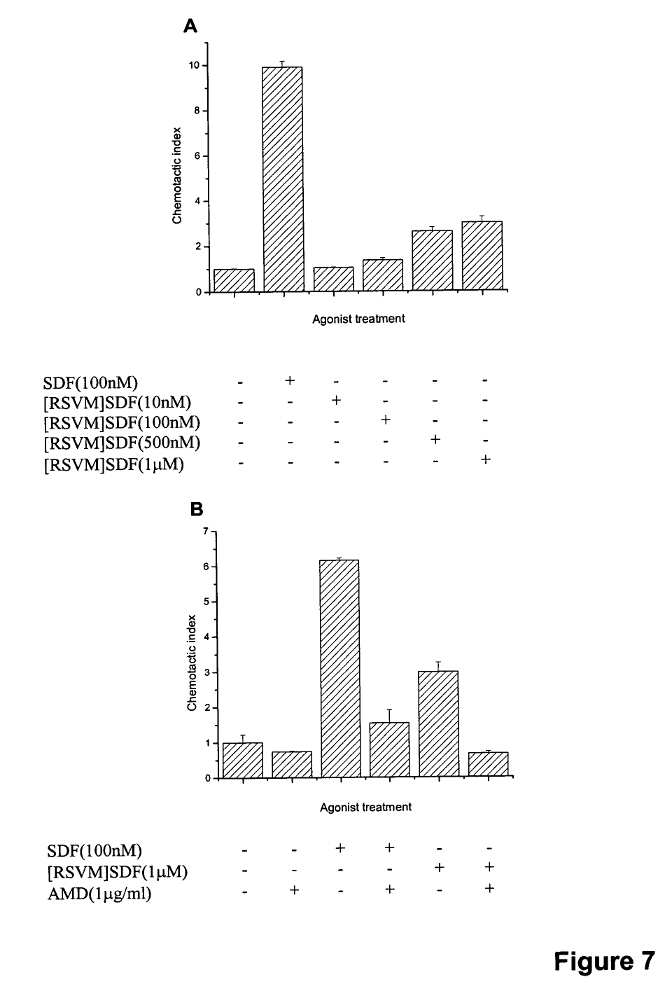

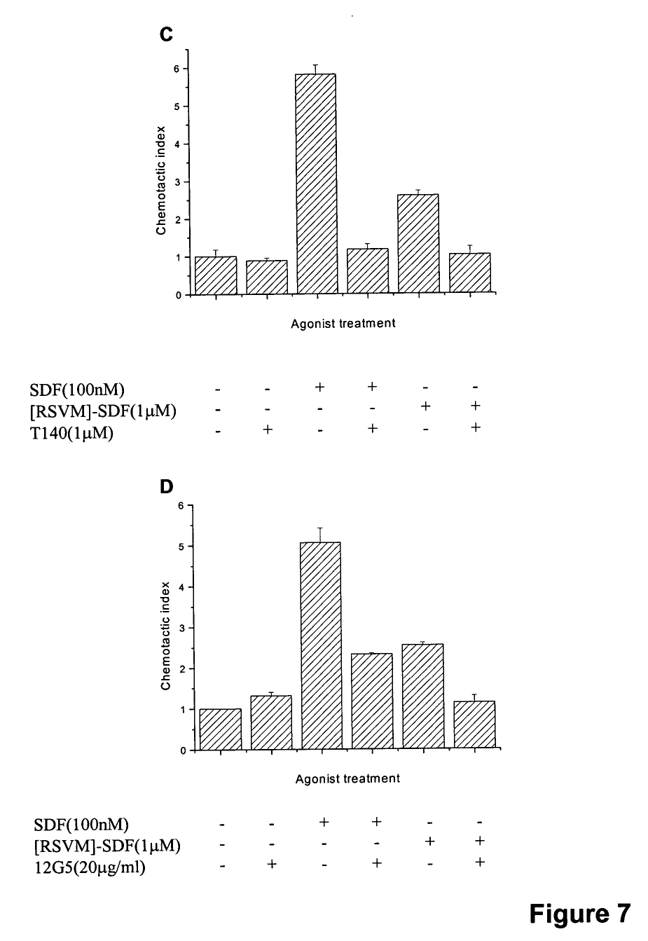

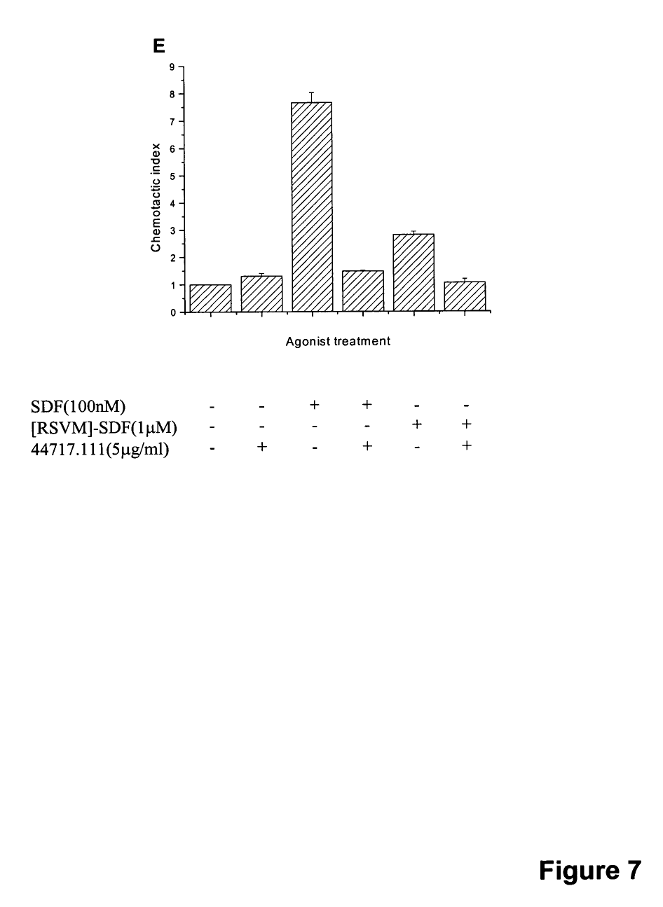

To determine the effect of the mutations in the context of the full-length SDF, we overexpressed and purified the two mutant SDF's from mammalian cells. The [ASLW]SDF-Fc mutant was inactive in chemotactic assays on CCRF-CEM cells and did not inhibit the activity of SDF-Fc on these cells at the concentrations tested (up to 2 μM) (data not shown). However, the [RSVM]SDF-Fc mutant displayed chemotactic activity on CCRF-CEM cells at ˜500 nM-1 μM (FIG. 7A). The efficacy of this full-length mutant was substantially less than wild type. Furthermore, this activity was completely inhibited by AMD3100, T140, 12G5 and 44717.111 (FIGS. 7B, 7C, 7D, and 7E) indicating that the full-length mutant has a similar binding site to CXCR4 as the wild-type protein.

Flow Cytometry.

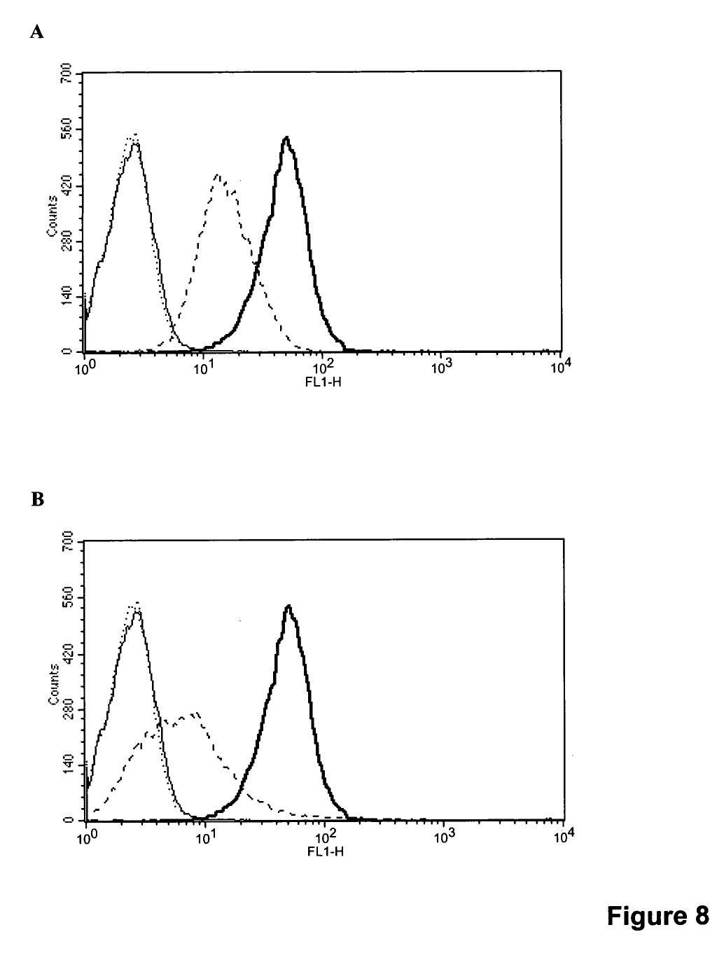

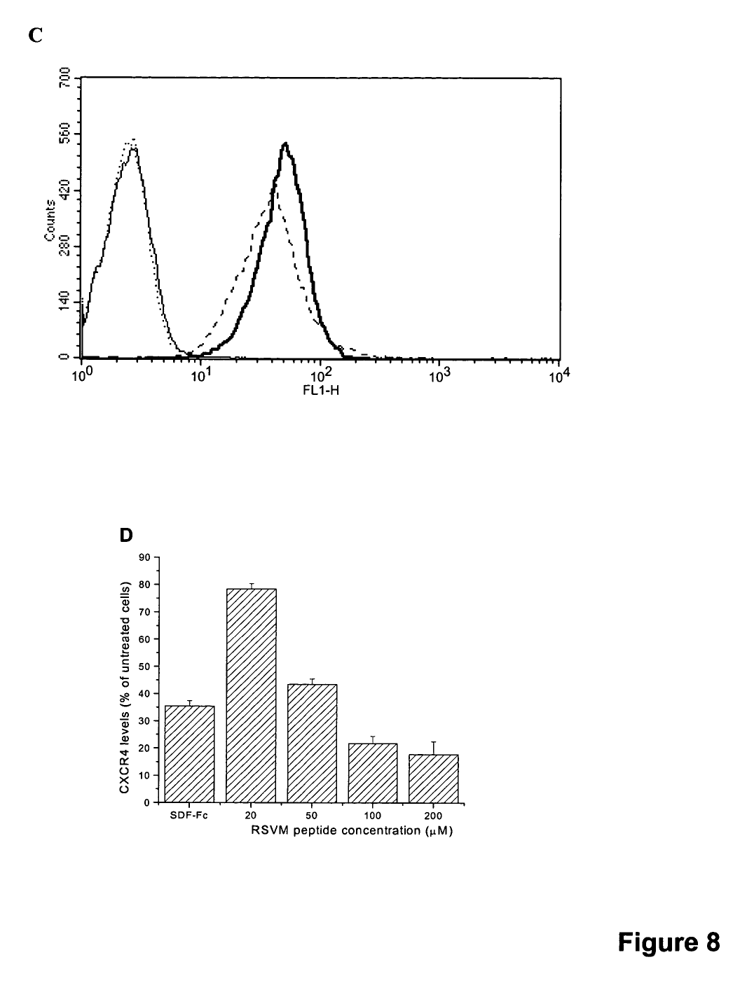

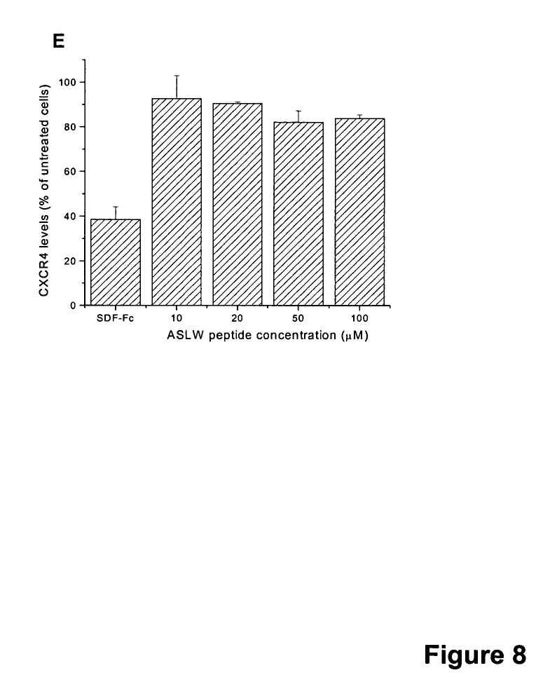

After CXCR4 activation, SDF-1 a induces receptor internalization (42). In order to gain insight on the differences in potency between the peptides and SDF-1α we examined their effect on surface levels of CXCR4 as detected using flow cytometry (and a FITC/12G5-based indirect staining procedure). As can be seen in FIG. 8A, SDF-Fc (1 μM) caused a clear reduction of the CXCR4 levels on the cell surface. The RSVM peptide had a similar effect (FIG. 8B) whereas ASLW caused only aminor decrease (FIG. 8C). The effect of RSVM was concentration-dependent (FIG. 8D). No significant concentration-dependent response was observed for ASLW (FIG. 8E). These data show that RSVM causes down-regulation of CXCR4 in a manner similar to SDF-Fc. On the contrary, ASLW does not induce a marked reduction in CXCR4 surface levels after cell activation, which may partially explain its superagonist activity.

Discussion

CXCR4 Activation.

The activation of CXCR4 and most chemokine receptors is believed to involve a two-step process. The first step involves the binding of SDF-1α to the CXCR4 N-terminus followed by activation of the receptor through interactions with the second extracellular loop (43). Each extracellular region of chemokine receptors possesses a cysteine. The four cysteines form two disulfides that are believed to bring the extracellular regions together into a compact structure with a stable binding site (44). Accordingly, the binding and activation sites are likely to be in close proximity. The activation of the receptor by the N-terminal region of SDF-1α results in an intracellular conformational change leading to an exchange of GDP for GTP in G proteins and dissociation of the G proteins from CXCR4 to initiate downstream signal transduction mechanisms.

Allosteric CXCR4 Agonists.

The phenomenon of allosteric agonism is relatively uncommon in the GPCR literature. Most of the available allosteric ligands are either antagonists or modulators/enhancers of the activity of the natural ligands without having by themselves agonist activity. It has been suggested that the relative lack of allosteric agonists in the GPCR ligand repertoire may reflect the bias of most screens with radioligand binding assays for the native (orthosteric) site (45). With the advent of functional screens, the identification of allosteric agonists may become more widespread and thus lead to novel therapeutic agents.

Based on the differences of IC₅₀s of AMD3100 on chemotactic and CXCR4 binding assays of SDF-1α as well as the biphasic binding behavior of [¹²⁵I]-SDF-1α on CXCR4-expressing cell lines, it has been proposed that AMD3100 and SDF-1α can bind to CXCR4 simultaneously (46). Mutagenesis experiments suggest that the second extracellular loop, transmembrane IV, or transmembrane VI of CXCR4 are critical for its interaction with AMD3100 (47,48). In principle, this allows novel agonists to bind to N-terminus or to other regions of the receptor that are not necessary for antagonist(s) interactions. Our results suggest that other binding sites exist for non-physiological agonists. These agonists were identified by screening a semi-randomized 17-mer library in a yeast strain expressing a functional CXCR4 receptor. The original, wild-type 17-mer has the same sequence as the first 17 amino acids of SDF-1α and had been shown previously to activate CXCR4 (13). Activation of CXCR4 in this yeast strain induces the pheromone response pathway, with the exception that growth arrest is replaced with cell proliferation due to a number of genetic modifications. As a result of these changes, only cells that secrete an agonist peptide can grow on the appropriate selective media. In contrast to the wild-type 17-mer peptide and SDF-1α, two agonist peptides were identified that are insensitive to the CXCR4 small molecule antagonists AMD3100 and T140, and to the neutralizing anti-CXCR4 monoclonal antibodies 12G5 and 44717.111 in CCRF-CEM cells. One peptide with a sequence of RVSM for the first four amino acids behaves as a partial agonist. The other peptide, ASLW, is a superagonist, displaying a chemotactic index that is greater than the maximum observed with SDF-Fc. SDF-Fc and RSVM peptide display a typical down-regulation of surface CXCR4 (FIGS. 8A, B and D). However, ASLW does not induce CXCR4 internalization (FIGS. 8C and E), which may explain the superagonist activity of this peptide due to continuous activation of the receptor. Although experiments with yeast strains clearly indicate that RSVM and ASLW are mediated by CXCR4 (FIG. 3), an alternative explanation in CCRF-CEM cells is that ASLW-induced chemotaxis may be mediated by another mechanism. This possibility remains to be further investigated.

When the same mutations are introduced into the full-length SDF-1α protein, one mutant, [ASLW]SDF-Fc, is inactive and the other, [RSVM]SDF-Fc, displays properties of a partial agonist that is sensitive to antibodies and small molecule antagonists (FIG. 7). The activity and pharmacological behavior of the [RSVM]SDF-Fc mutant indicates that it binds to the natural agonist site. Furthermore the EC₅₀ increases from 1-10 nM for SDF-1α (31) to >1 μM for the wild-type 17-mer peptide (13). Taken together these data indicate that the rest of the protein contributes substantially to receptor binding. The results also suggest that the mutations at the N-terminal region of the two peptides alter the binding site on the receptor.

Drug Development.

Our studies present proof of principle for allosteric agonism on CXCR4 that makes possible the screening of non-peptide libraries for such activity. In order to optimize their clinical usefulness, the RSVM and ASLW peptides should be modified. The potency and chemical stability of these molecules should be increased and their size reduced. There are a number of approaches to accomplish these goals. One of them consists of identifying the minimal peptide sequence that retains allosteric agonist activity followed by further randomization to improve potency. The yeast CXCR4 expression system can be used to pursue these goals. The chemical stability of the peptide ligands can be increased by synthesis of peptidomimetics based on the most potent sequences.

Therapeutic Implications.

Current state-of-the-art therapy of HIV infection involves combination of agents that act either at the reverse transcriptase or protease step of HIV-1 replication, termed highly active antiretroviral therapy (HAART). These agents did not show any serious toxicity in clinical trials. However, lipodystrophy and other metabolic complications were observed after widespread use of HAART in patients (49). The chemokine receptors CXCR4 and CCR5 are the two major co-receptors used by HIV-1 to infect cells after the HIV-1 surface protein gp120 engages CD4 (50). This induces a conformational change that leads to the insertion of the viral protein gp41 into the cell membrane. This results in the release of virion contents into the cell. The initial infection by HIV-1 usually involves M-tropic strains that utilize the chemokine receptor CCR5 and are associated with the asymptomatic stage of the disease. Individuals lacking functional CCR5 due to a 32 base pair deletion in both alleles are highly resistant to infection by HIV-1 (51). The evolution from M-tropic strains to T-tropic strains involves mutations mainly in the V3 loop of gp120, which changes the co-receptor specificity from CCR5 to CXCR4 (52). The change to T-tropic strains leads to a decrease in T-cells and all the symptoms associated with full-blown AIDS. Agents that can prevent the transition or induce the reversion to CCR5-utilizing strains may decrease the morbidity and mortality of this infectious disease. However, therapeutic use of CXCR4 antagonists can be expected to have adverse effects due to the CXCR4-mediated trafficking of lymphocytes, monocytes, and hematopoietic stem cells (53,54). The CXCR4 antagonists AMD3100 and ALX40-4C have been used in clinical trials (55,56). The further development of AMD3100 was discontinued due to cardiac toxicity. This toxicity is not general for all CXCR4 antagonists since in the case of ALX40-4C administration no major adverse effects were reported (56). It should be noted that any CXCR4 antagonist that is approved for clinical use would be used chronically. Adverse effects that are not observed during clinical trials may be manifested over long-term use as in the case of HAART.

T20 is a synthetic peptide corresponding to a region of HIV-1 gp41 that blocks viral fusion to cell membranes (57). It is currently in phase III clinical trials without evidence of any serious systemic toxicity (55). Interestingly, strong in vitro synergy has been observed between T-20 and AMD3100 (58) suggesting that a possible combination of gp41 and CXCR4 inhibitors may provide significant therapeutic advantages in HIV treatment. This possibility further establishes the need for an approach that minimizes the toxic effects of AMD3100 and other CXCR4 antagonists. In this context, allosteric CXCR4 agonists could maintain essential receptor function and allow the clinical combination of CXCR4 antagonists with other anti-HIV agents that have a different mechanism of action.

Acknowledgements

This work was supported by grants GM35208 (to A.H.) and A143838 (to E.L.) from the NIH.

### REFERENCES

1. Gether, U. (2000) Endocr Rev 21(1), 90-113.
2. Ulloa-Aguirre, A., Stanislaus, D., Janovick, J. A., and Conn, P. M. (1999) Arch Med Res 30(6), 420-35.
3. Knoflach, F., Mutel, V., Jolidon, S., Kew, J. N., Malherbe, P., Vieira, E., Wichmann, J., and Kemp, J. A. (2001) Proc Natl Acad Sci U S A 98(23), 13402-7.
4. Nagasawa, T., Hirota, S., Tachibana, K., Takakura, N., Nishikawa, S., Kitamura, Y., Yoshida, N., Kikutani, H., and Kishimoto, T. (1996) Nature 382(6592), 635-8.
5. Tachibana, K., Hirota, S., Iizasa, H., Yoshida, H., Kawabata, K., Kataoka, Y., Kitamura, Y., Matsushima, K., Yoshida, N., Nishikawa, S., Kishimoto, T., and Nagasawa, T. (1998) Nature 393(6685), 591-4.
6. Zou, Y. R., Kottmann, A. H., Kuroda, M., Taniuchi, I., and Littman, D. R. (1998) Nature 393(6685), 595-9.
7. Feng, Y., Broder, C. C., Kennedy, P. E., and Berger, E. A. (1996) Science 272(5263), 872-7.
8. Crump, M. P., Gong, J. H., Loetscher, P., Rajarathnam, K., Amara, A., Arenzana-Seisdedos, F., Virelizier, J. L., Baggiolini, M., Sykes, B. D., and Clark-Lewis, I. (1997) Embo J 16(23), 6996-7007.
9. Dealwis, C., Fernandez, E. J., Thompson, D. A., Simon, R. J., Siani, M. A., and Lolis, E. (1998) Proc Natl Acad Sci USA 95(12), 6941-6.
10. Fernandez, E. J., and Lolis, E. (2002) Annu Rev Pharmacol Toxicol 42, 469-99
11. Clark-Lewis, I., Dewald, B., Loetscher, M., Moser, B., and Baggiolini, M. (1994) J Biol Chem 269(23), 16075-81.
12. Baggiolini, M., Dewald, B., and Moser, B. (1997) Annu Rev Immunol 15, 675-705
13. Loetscher, P., Gong, J. H., Dewald, B., Baggiolini, M., and Clark-Lewis, I. (1998) J Biol Chem 273(35), 22279-83.
14. King, K., Dohlman, H. G., Thorner, J., Caron, M. G., and Lefkowitz, R. J. (1990) Science 250(4977), 121-3.
15. Price, L., Kajkowski, E., Hadcock, J., Ozenberger, B., and Pausch, M. (1995) Molecular Cellular Biology 15, 6188-95
16. Price, L., Strnad, J., Pausch, M., and Hadcock, J. (1996) Molecular Pharmacology 50, 829-37
17. Erickson, J. R., Wu, J. J., Goddard, J. G., Tigyi, G., Kawanishi, K., Tomei, L. D., and Kiefer, M. C. (1998) J Biol Chem 273(3), 1506-10.
18. Klein, C., Paul, J. I., Sauve, K., Schmidt, M. M., Arcangeli, L., Ransom, J., Trueheart, J., Manfredi, J. P., Broach, J. R., and Murphy, A. J. (1998) Nat Biotechnol 16(13), 1334-7.
19. Baranski, T. J., Herzmark, P., Lichtarge, O., Gerber, B. O., Trueheart, J., Meng, E. C., Iiri, T., Sheikh, S. P., and Bourne, H. R. (1999) J Biol Chem 274(22), 15757-65.
20. Geva, A., Lassere, T. B., Lichtarge, O., Pollitt, S. K., and Baranski, T. J. (2000) J Biol Chem 275(45), 35393-401.
21. Erlenbach, I., Kostenis, E., Schmidt, C., Hamdan, F. F., Pausch, M. H., and Wess, J. (2001) J Neurochem 77(5), 1327-37.
22. Erlenbach, I., Kostenis, E., Schmidt, C., Serradeil-Le Gal, C., Raufaste, D., Dumont, M. E., Pausch, M. H., and Wess, J. (2001) J Biol Chem 276(31), 29382-92.
23. Zhang, W. B., Navenot, J. M., Haribabu, B., Tamamura, H., Hiramatu, K., Omagari, A., Pei, G., Manfredi, J. P., Fujii, N., Broach, J. R., and Peiper, S. C. (2002) J Biol Chem 277(27), 24515-21.
24. Ausubel, F., Brent, R., Kingston, R., Moore, D., Siedman, J., Smith, J., and Struhl, K. (eds) (1993) Current Protocols in Molecular Biology, John Wiley and Sons
25. Manfredi, J. P., Klein, C., Herrero, J. J., Byrd, D. R., Trueheart, J., Wiesler, W. T., Fowlkes, D. M., and Broach, J. R. (1996) Mol Cell Biol 16(9), 4700-9.
26. Agatep, R., Kirkpatrick, R., Parchaliuk, D., Woods, R., and Gietz, R. (1998) Technical Tips Online
27. Ehrhard, K. N., Jacoby, J. J., Fu, X. Y., Jahn, R., and Dohlman, H. G. (2000) Nat Biotechnol 18(10), 1075-9.
28. Brown, A. J., Dyos, S. L., Whiteway, M. S., White, J. H., Watson, M. A., Marzioch, M., Clare, J. J., Cousens, D. J., Paddon, C., Plumpton, C., Romanos, M. A., and Dowell, S. J. (2000) Yeast 16(1), 11-22.
29. Ueda, H., Siani, M. A., Gong, W., Thompson, D. A., Brown, G. G., and Wang, J. M. (1997) J Biol Chem 272(40), 24966-70.
30. Bridger, G. J., Skerlj, R. T., Thornton, D., Padmanabhan, S., Martellucci, S. A., Henson, G. W., Abrams, M. J., Yamamoto, N., De Vreese, K., Pauwels, R., and et al. (1995) J Med Chem 38(2), 366-78.
31. Hesselgesser, J., Liang, M., Hoxie, J., Greenberg, M., Brass, L. F., Orsini, M. J., Taub, D., and Horuk, R. (1998) J Immunol 160(2), 877-83.
32. Chang, F., and Herskowitz, I. (1990) Cell 63(5), 999-1011.
33. Luo, Z., Zhou, N., Luo, J., Hall, J. W., and Huang, Z. (1999) Biochem Biophys Res Commun 263(3), 691-5.
34. Donzella, G. A., Schols, D., Lin, S. W., Este, J. A., Nagashima, K. A., Maddon, P. J., Allaway, G. P., Sakmar, T. P., Henson, G., De Clercq, E., and Moore, J. P. (1998) Nat Med 4(1), 72-7.
35. Tamamura, H., Xu, Y., Hattori, T., Zhang, X., Arakaki, R., Kanbara, K., Omagari, A., Otaka, A., Ibuka, T., Yamamoto, N., Nakashima, H., and Fujii, N. (1998) Biochem Biophys Res Commun 253(3), 877-82.
36. Arakaki, R., Tamamura, H., Premanathan, M., Kanbara, K., Ramanan, S., Mochizuki, K., Baba, M., Fujii, N., and Nakashima, H. (1999) J Virol 73(2), 1719-23.
37. McKnight, A., Wilkinson, D., Simmons, G., Talbot, S., Picard, L., Ahuja, M., Marsh, M., Hoxie, J. A., and Clapham, P. R. (1997) J Virol 71(2), 1692-6.
38. Biard-Piechaczyk, M., Robert-Hebmann, V., Richard, V., Roland, J., Hipskind, R. A., and Devaux, C. (2000) Virology 268(2), 329-44.
39. Endres, M. J., Clapham, P. R., Marsh, M., Ahuja, M., Turner, J. D., McKnight, A., Thomas, J. F., Stoebenau-Haggarty, B., Choe, S., Vance, P. J., Wells, T. N., Power, C. A., Sutterwala, S. S., Doms, R. W., Landau, N. R., and Hoxie, J. A. (1996) Cell 87(4), 745-56.
40. Brelot, A., Heveker, N., Adema, K., Hosie, M. J., Willett, B., and Alizon, M. (1999) J Virol 73(4), 2576-86.
41. Labrosse, B., Brelot, A., Heveker, N., Sol, N., Schols, D., De Clercq, E., and Alizon, M. (1998) J Virol 72(8), 6381-8.
42. Orsini, M. J., Parent, J. L., Mundell, S. J., Benovic, J. L., and Marchese, A. (1999) J Biol Chem 274(43), 31076-86.

1. A method of using an allosteric CXCR4 agonist in AIDS treatment, wherein the method is characterized by side effects that are less adverse than those associated with the use of CXCR4 antagonists in AIDS treatment.

2. The method of claim 1, wherein the adverse effects include at least one of hematopoietic and cardiac effects.

3. A method of using an Allosteric CXCR4 agonist, wherein the method includes inhibiting cell entry and replication of HIV strains that have become resistant to CXCR4 ligands.

4. The method of claim 3, wherein the CXCR4 ligands include at least one of AMD3100 and T140.

5. A method of using an allosteric CXCR4 agonist in breast cancer treatment, wherein the method is characterized by a toxicity that is lower than that associated with the use of CXCR4 antagonists in breast cancer treatment.
